(19) United States
(12) Patent Application Publication
(10) Pub. No.: US 2026/0335670 A1
(43) Pub. Date: Oct. 29, 2026

(54) ILLUMINATION DEVICE FOR PHOTODYNAMIC THERAPY, METHOD FOR TREATING A SKIN DISEASE AND METHOD FOR OPERATING AN ILLUMINATION DEVICE

(71) Applicant: Biofrontera Pharma GmbH, Leverkusen (DE)

(72) Inventors: Hermann LÜBBERT, Leverkusen (DE); Markus OSTERLOH, Essen (DE); Ben NOVAK, Leverkusen (DE); Montserrat FOGUET ROCA, Leverkusen (DE); Monika KOKOSZKA, Leverkusen (DE); Wiebke MEYER-WENDT, Leverkusen (DE); Martin LÜBBERT, Troisdorf (DE)

(21) Appl. No.: 19/122,218

(22) PCT Filed: Oct. 20, 2022
(86) PCT No.: PCT/EP2022/079298

§ 371 Date: Apr. 17, 2025

(51) Int. Cl. A61N 5/06; A61N 5/00

(52) U.S. Cl. CPC A61N 5/062; A61N 5/0616; A61N 2005/005; A61N 2005/0628; A61N 2005/0632

(57) ABSTRACT

Illumination device for photodynamic therapy, the illumination device comprising at least two electromagnetic radiation emitting units, the at least two electromagnetic radiation emitting units comprising at least one electromagnetic radiation source, the electromagnetic radiation source being configured to generate radiation for the irradiation of a region of an irradiation object in an illumination session, wherein the irradiation object is to be arranged at an object location, wherein the object location is arranged at a distance relative to a radiation output region of the radiation emitting unit through which the radiation generated by the at least one electromagnetic radiation source exits the radiation emitting unit during operation of the illumination device.

This disclosure relates to an illumination device for photodynamic therapy. Furthermore, the disclosure relates to a method for operating an illumination device, a method for treating skin disease and a computer program product as well as a computer-readable medium.

Photodynamic therapy (PDT) has been widely studied and several approaches have been used successfully for treatment. In general, there are three requirements for PDT: a photosensitizer, molecular oxygen and light of a specific wavelength. For dermatological PDT usually a prodrug, for example aminolevulinic acid (ALA), is topically applied to the skin. Subsequently, the prodrug is then converted by the cells, e.g. by neoplastic cells, into the actual photosensitizer. The molecular mechanism of action in PDT is based on cellular ALA uptake, synthesis and accumulation of the photosensitizer, which can be excited by light of specific wavelengths leading to the formation of reactive oxygen species (ROS), upon the presence of oxygen. The ROS can initiate cell death, e.g. in the form of apoptosis, necrosis and autophagy.

However, one of the major issues that hinder broad acceptance of PDT by patients is the relatively high amount of pain perceived by the patients during the illumination which ranges from mild inconvenience to severe pain to a point where the treatment has to be aborted. In addition, although PDT is a highly effective treatment method, reoccurrence of some diseases like actinic keratosis is common and thus patients often, although having been successfully treated, later develop different lesions at different skin regions and again require medical intervention. Moreover, some patients are not completely cured after a single PDT session and require a second session. If the first PDT they received was very painful the chances of beginning or completing a second PDT are small despite the fact that it offers supreme efficacy compared to other therapy options. As a result, the acceptance of many patients to undergo treatment or re-treatment decreases. This of course has great negative implications for an individual PDT and PDT as a whole.

Moreover, PDT efficacy is also limited by any of the involved factors, i.e. photosensitizer, oxygen, and light. Reduced availability of any of these factors may hamper with ROS formation. Optimized pharmaceutical forms, pretreatments and incubation modalities can ensure proper and abundant deposition of the photosensitizer. Still, light has to reach a molecule in sufficient quantities and oxygen needs to be present as an energy acceptor.

In particular, the light of the illumination at the appropriate wavelength to activate the respective photosensitizer needs to be made available at a sufficient dose. For topical applications, a frequently used photosensitizer is protoporphyrin IX (PpIX), mostly produced in skin cells by application of a precursor molecule, such as ALA. PpIX can be activated by light of a variety of different wavelengths of which red (approx. 635 nm), blue (approx. 420 nm), yellow (approx. 542 nm) or green (approx. 506 nm) light are most frequently used. Generally, a radiation dose received by a target, e.g. the treated skin, depends on three main factors. The irradiance provided by the illumination device, the distance between the target region and the illumination device, and the duration of the illumination.

The current practice is to apply the entire radiation dose within a short interval (for example ranging from 7 to 12 minutes with red light or 15-20 minutes with blue light). Usually, this approach is limited by the occurrence of pain in the patient. Moreover, photobleaching of the photosensitizer may occur to a greater extent at higher light intensities and may limit the treatment efficiency. Photobleaching describes the effect that the photosensitizer is inactivated by permanent disruption of its chemical structure, e.g. by cleavage of covalent bonds. This photobleaching effect may coincide with temporal oxygen depletion in the target tissue due to a massive initial reaction. This leads to a rapid decrease in oxygen, which is required for ROS formation. All photobleaching that occurs during the phase where oxygen is limited is likely to be unproductive, as it yields fewer cytotoxic singlet oxygen.

It should be noted that the statements above should not be construed as being admitted prior art. They are only made to illustrate the background of the presently disclosed concepts and may not have been made available to the public yet.

One object to be achieved is to provide an improved illumination device for photodynamic therapy. A further object to be achieved is to provide a method for treating a skin disease in which such an illumination device is used. A further object to be achieved is to provide a method for operating such an illumination device.

The respective object may, inter alia, be achieved by the subject matter of claims 1 and 44 and 47. Advantageous embodiments and further developments are the subject of the dependent claims. However, further advantageous concepts may be disclosed herein besides the ones which are currently claimed.

Firstly, the illumination device is specified in more detail.

According to at least one embodiment, the illumination device comprises at least one electromagnetic radiation emitting unit (herein also called “radiation emitting unit”). “At least one” means that the illumination device may comprise one or more radiation emitting units, e.g. two or more radiation emitting units. All features which are in the following disclosed for one radiation emitting unit are likewise disclosed for all other radiation emitting units of the illumination device or only for some radiation emitting units of the device. The radiation or light emitted by the radiation emitting unit is, for example, radiation in the visible wavelength region.

According to at least one embodiment, the electromagnetic radiation emitting unit comprises at least one electromagnetic radiation source (herein also called “radiation source”). This means, that the electromagnetic radiation emitting unit may comprise one or more electromagnetic radiation emitting sources. All features which are in the following disclosed for one electromagnetic radiation source are likewise disclosed for all electromagnetic radiation sources of the radiation emitting unit or of the illumination device or only some radiation sources.

According to at least one embodiment, the at least one radiation source is an optoelectronic component, for example a light emitting diode (LED) and/or a surface mountable component. All features disclosed so far and in the following for the at least one radiation source are likewise disclosed for all or some radiation sources of the illumination device.

According to at least one embodiment the radiation sources may emit radiation of the same or similar peak wavelengths, e.g. radiation of the same colour, e.g. red light (635 nm±4 nm), blue light (420 nm±4 nm), yellow light (542 nm±4 nm) or green light (506 nm±4 nm).

According to at least one embodiment, the emission spectrum of the optoelectronic component has a peak wavelength in one of the following ranges: 634 nm±4 nm, 634 nm±5 nm, 635 nm±4 nm, 635 nm±5 nm, 542 nm±4 nm, 542 nm±5 nm, 506 nm±4 nm, 506 nm±5 nm, 420 nm±4 nm, 420 nm±5 nm.

Particularly, this peak wavelength is obtained at an operating temperature of the optoelectronic component below 50 °C, for example at 25 °C, and at operating currents between 100 mA and 1000 mA, inclusive. The half-band width of the spectrum is, e.g. at least 10 nm and/or at most 20 nm, e.g. 16 nm.

According to at least one embodiment, the electromagnetic radiation source is configured to generate radiation for the irradiation of a region (herein also called “irradiation region”) of an irradiation object in an illumination session. Thus, the electromagnetic radiation source is an element of the electromagnetic radiation emitting unit which produces the radiation emitted by the radiation emitting unit. The irradiation object is, for example, a mammal, e.g. a human. The irradiation region of the irradiation object is, for example, a skin region of the human. An illumination session lasts, for example, at most 120 minutes, at most 80 minutes, at most 60 minutes, at most 40 minutes, at most 30 minutes, e.g. 20 minutes or less.

According to at least on embodiment, the duration of the entire illumination session is less than or equal to one of the following values: 25 min, 24 min, 23 min, 22 min, 21 min, 20 min, 19 min, 18 min, 17 min, 16 min, 15 min, 14 min, 13 min. Session durations up to 25 minutes, e.g. 22 minutes, are usually accepted by users. Additionally or alternatively, the duration of the entire illumination session is greater than or equal to one of the following values: 10 min, 11 min, 12 min, 13 min. The duration of the session may be between 10 min and 25 min, for example, e.g. 18 minutes or 22 minutes.

According to at least one embodiment, the irradiation object is to be arranged at a predetermined object location, e.g. during the illumination session. The predetermined object location preferably is a space region or a point in space spaced apart from the illumination device and/or the radiation emitting unit.

During intended operation, the region of the irradiation object to be irradiated is located at or inside, for example completely inside, the predetermined object location. The region of the irradiation object to be irradiated is, during intended operation, also spaced apart from the illumination device and/or the radiation emitting unit.

According to at least one embodiment, the predetermined object location is arranged at a distance relative to a radiation output region of the radiation emitting unit through which the radiation generated by the at least one electromagnetic radiation source exits the radiation emitting unit during operation of the illumination device. A distance between two objects is herein defined as the shortest connection between the two objects. For example, during the illumination session, the distance between the predetermined object location and the output region is greater than or equal to one of the following values: 50 mm, 60 mm, 70 mm, 80 mm. Additionally or alternatively, the distance may be less than or equal to one of the following values: 800 mm, 700 mm, 600 mm, 500 mm, 400 mm, 300 mm, 100 mm, 80 mm. Likewise, the distance between the irradiation region of the irradiation object and the radiation output region may have these values during the illumination session.

The radiation dose may be greater than or equal to one of the following values when the irradiation object is arranged at the object location during the illumination session: 30 J/cm², 35 J/cm², 37 J/cm².

Alternatively, or additionally, the radiation dose may be less than or equal to one of the following values when the irradiation object is arranged at the object location during the illumination session: 45 J/cm², 40 J/cm², 37 J/cm². The mean or maximum irradiance may be greater or equal to one of the following values when the irradiation object is arranged at the object location during the illumination session: 25 mW/cm², 40 mW/cm², 50 mW/cm². Alternatively, or additionally, the mean or maximum irradiance may be smaller or equal to one of the following values when the irradiation object is arranged at the object location during the illumination session: 75 mW/cm², 65 mW/cm², 60 mW/cm². For example, the mean or maximum irradiance when the irradiation object is arranged at the object location during the illumination session is 62 mW/cm²+/−1 mW/cm². The values above particularly hold at least for red light, e.g. with a peak wavelength at 635 nm at 25 °C.

A sufficient radiation dose is one of the key requirements to successfully carry out PDT. However, when choosing the radiation dose, the maximum tolerable level of pain for the patient should also be considered. The range between a radiation dose of 30 and 45 J/cm², in particular a radiation dose of 37 J/cm², may be considered the best compromise between adequate treatment efficiency and pain burden, e.g. when using red light. Additionally, or alternatively, a mean or maximum irradiance between 25 mW/cm² and 75 mW/cm², particularly a mean or maximum irradiance of 62 mW/cm², may be considered the best compromise between adequate treatment efficiency and pain burden, e.g. when using red light.

According to at least one embodiment, the illumination device is configured to irradiate the region of the irradiation object with a predetermined radiation dose during the illumination session. The radiation dose may be greater than or equal to one of the following values when the irradiation object is arranged at the object location during the illumination session: 8 J/cm², 9 J/cm², 10 J/cm². Alternatively, or additionally, the radiation dose may be less than or equal to one of the following values when the irradiation object is arranged at the object location during the illumination session: 12 J/cm², 11 J/cm², 10 J/cm². The values above particularly hold at least for blue light.

The range between a radiation dose of 8 J/cm² and 12 J/cm², in particular a radiation dose of 10 J/cm², may be considered the best compromise between adequate treatment efficiency and pain burden, e.g. when using blue light.

As mentioned above, pain reduction is of crucial interest to increase acceptance levels of PDT treatment as a whole, thus increasing the use of this superior treatment. One important step to reach this goal is to increase the efficacy of PDT, e.g. by improving the homogeneity or uniformity of the irradiation of the skin, respectively. In this way, the probability that one or only a few sessions are sufficient to treat the affected skin region, is increased. Moreover, the probability of photobleaching in certain regions is also reduced if the homogeneity is increased.

The term “homogeneity” has to be understood as special homogeneity, i.e. a uniform special distribution of radiation on an irradiation region, and as homogeneity in radiation power and/or wavelength, i.e. the radiation power and/or the wavelength of the radiation is homogenous over time during an illumination session.

With the illumination device disclosed herein, an improvement in the radiation dose received by the target, particularly an improvement in terms of the homogeneity of irradiation, is, inter alia, achieved.

Further aspects of the illumination device leading to an improved irradiation effect and experience of an irradiation object will be explained in more detail below.

According to at least one embodiment, the radiation emitting unit comprises a plurality of radiation sources arranged on a common radiation source carrier. The radiation source carrier is part of the radiation emitting unit. The radiation source carrier is, for example, a contiguously formed carrier. The carrier may have a continuous surface on which a plurality of radiation sources is arranged. The radiation source carrier may be self-supporting. It expediently carries the radiation sources arranged on it. The carrier may be a circuit board.

According to at least one embodiment, the radiation sources on the radiation source carrier are grouped into a plurality of groups, wherein the radiation sources of each group are arranged in a regular group pattern, wherein at least two groups of the plurality of groups have different group patterns. In a regular pattern, the primitive translation vectors do not change across the entire group. Different group patterns are, for example, different from each other in terms of one or two primitive translation vectors. For example, each radiation source on the radiation source carrier is assigned to one group.

According to at least one embodiment, at least two groups, particularly all groups, have the same number of radiation sources.

According to at least one embodiment, each group has a plurality of radiation sources, for example between four and 40, inclusive, or between ten and 25, inclusive.

According to at least one embodiment, the illumination device comprises an electronic control unit which is configured to control the operation of the illumination device, wherein the respective radiation emitting unit is operatively coupled to the electronic control unit.

According to at least one embodiment, the illumination device is configured to consider temperature dependent variations in the wavelength of the radiation emitted by the radiation sources and/or temperature dependent variations in the optical output power, e.g. in the radiation power, for adjusting parameters of the illumination session in order to irradiate the predetermined radiation dose onto the irradiation region.

With changes in temperature of the radiation source(s) and/or of the environment in which the illumination device is operating, the radiation emission properties, e.g. the wavelength, may shift. For example, the irradiation and the peak emission wavelength may increase or decrease depending on the temperature and the characteristics, e.g. the type, of the radiation source(s). A shift of the wavelength could move the emission spectrum away from a maximum in the absorption spectrum which would in turn have the similar results as a shortage of delivered energy. Due to an insufficient amount of absorbed energy, the formation of cytotoxic singlet oxygen is also decreased with the same negative consequences for the treatment success.

Temperature changes in the environment in which the radiation is generated may therefore have an effect on the wavelength of the radiation emitted and as such on the success rate of the treatment. By considering the temperature dependent variations in the wavelength of the radiation emitted by the radiation source, changes in temperature in the operation environment caused e.g. by different season of a year, e.g. in winter and summer, different time of the day, e.g. early morning and midafternoon, and/or dependent on geographical location of the illumination device are considered and taken into account during operation of the illumination device, e.g. during an illumination session, guaranteeing thereby a better treatment.

Furthermore, this guarantees also a more flexible use of the illumination device, in particular location-wise. The illumination device may be moved within a location, e.g. within a room, or within a building complex, without requiring consideration of temperature changes within the different locations by a user and hence delivering uniform and homogeneous treatment results, independently from the environment in which the illumination session occurs.

Hence, it is advantageous to take into consideration temperature dependent variations in the wavelength of the radiation emitted by the radiation sources.

Additionally, or alternatively, also temperature dependent variation in the optical output power are taken into consideration. The optical output power of a radiation source, e.g. of a LED, is also temperature dependent and as such prone to temperature dependent variations. Adjusting parameters of the illumination device depending on the temperature-dependent variations in the optical output can compensate for these variations and hence guarantee a more efficient and effective treatment.

According to at least one embodiment, the illumination device comprises at least one temperature sensor, which is configured to measure a temperature, e.g. a temperature characteristic for the temperature of the radiation source(s). The at least one temperature sensor may be operatively connected to the electronic control unit to provide temperature data to the electronic control unit. The electronic control unit may be configured to adjust the operation of the illumination device based on the temperature data to ensure that the predetermined radiation dose is delivered to the irradiation object.

The at least one temperature sensor may be located at or near a radiation source. In case of more radiation sources, a temperature sensor at each radiation source may be provided or just one or more temperature sensor(s) at one or more of the radiation sources, e.g. at least one or only one temperature sensor per radiation emitting unit.

In case of more temperature sensors, each temperature sensor may be operatively connected to the electronic control unit to provide temperature values. The electronic control unit may average some or all of the different values such as to obtain a single temperature data value for some or all of the radiation sources.

The temperature sensor may be physically connected to the electronic control unit, e.g. through a cable connection, or wirelessly operatively connected to the electronic control unit, e.g. via Bluetooth, via Wi-Fi or similar.

The at least one temperature sensor may continuously provide temperature data during operation of the illumination device. The temperature sensor may provide regular temperature values during operation of the illumination device, such as with a frequency of every second, every two seconds, every three seconds, every four seconds, every five seconds or less frequent. In other words, the temperature sensor may be polled with an appropriate frequency.

According to at least one embodiment, the illumination device comprises a radiation sensor which is arranged to receive radiation emitted from the illumination device in order to generate radiation data which is characteristic for a wavelength shift of the peak wavelength of the radiation source, wherein the electronic control unit is configured to adjust the operation of the illumination device based on the radiation data to ensure that the predetermined radiation dose is delivered to the irradiation region, e.g. during the illumination session.

If for example, the peak wavelength of the radiation source has a shifted such as not to guarantee an optimal radiation dose of the irradiation region of an irradiation object, the electronic control unit may adjust the operation such as to e.g. prolong or shorten the irradiation period of the irradiation object and/or increase or decrease the wavelength of the radiation emitted by the radiation emitting units.

According to at least one embodiment, the radiation received by the radiation sensor is radiation reflected by the irradiation object. The radiation may be radiation generated by the radiation sources.

Radiation sensors may be used similarly as temperature sensors to adjust the operation of the illumination device by the electronic control unit. In particular, the illumination device may comprise one or more radiation sensor(s).

In case of more radiation sensors, each radiation sensor may be operatively connected to the electronic control unit to provide radiation data. The electronic control unit may average the different values such as to obtain a single radiation data value.

The radiation sensor may be physically connected to the electronic control unit, e.g. through a cable connection, or wirelessly operatively connected, e.g. via Bluetooth, via Wi-Fi or similar.

The radiation sensor may continuously provide radiation data during operation of the illumination device. The radiation sensor may provide regular data during operation device, such as every second, every two seconds, every three seconds, every four seconds, every five seconds or less frequent. In other words, the radiation sensor may be polled with an appropriate frequency.

One or more radiation sensors may be located at or near a radiation source and may be configured to receive the radiation reflected from an irradiation object. The data is then provided to the electronic control unit which based on the received information may extrapolate how much radiation has been absorbed by the irradiation object and may adjust the radiation dose accordingly.

According to at least one embodiment, the operation of the illumination device is adjusted by using one of, an arbitrary combination of or all of the following measures:
varying, e.g. increasing or decreasing, the distance between the respective radiation emitting unit and the irradiation object,
adjusting, e.g. increasing or decreasing, the radiation power emitted by the respective radiation emitting unit, and/or
adjusting, e.g. increasing or decreasing, a duration of the illumination session.

The adjustments may vary the radiation power impinging on the irradiation object.

Hence, the operation of the illumination device and consequently the irradiation of the irradiation object, e.g. a patient, is optimized, e.g. to compensate for variations in the peak wavelength or the radiation power. As such greater controllability and precise irradiation of an irradiation object can be achieved.

According to at least one embodiment, the at least two radiation emitting units are connected to a common support via a mechanical connection system.

According to at least one embodiment the common support of the illumination device is a portion of the illumination device substantially extending perpendicular to the floor, e.g. extending along a longitudinal axis perpendicular to the floor. The common support may further comprises a foot element to hold the illumination device on the ground. Additionally, the foot element may comprise wheels in order to allow the movability of the whole illumination device.

According to at least one embodiment, the radiation emitting units are movably, e.g. pivotally, connected to one another.

According to at least one embodiment, the mechanical connection system comprises a connection arm, which is rigidly and/or axially connected to the at least two radiation emitting units, which may be movable relative to one another and connected with one another. The connection arm therefore connects the at least two radiation emitting units to the common support. “Axially” connected may mean that the connection prevents axial movement of the radiation unit to which the arm is connected and the location of the connection to the arm is prevented. Rotational movement may be allowed.

According to at least one embodiment, the connection arm is U-shaped, e.g. horseshoe shaped, V-shaped or C-Shaped.

According to at least one embodiment, the connection arm is configured to be movable from a first position into a second position, wherein the U-shape, e.g. the horseshoe shape, V-shape or C-shape of the connection arm in the first position is narrower than the U-shape, e.g. the horseshoe shape, V-shape or C-shape of the connection arm in the second position. A narrower U-, V-or C-shape in a first position is defined as a U-, V-or C-shape in which the distance between the two ends of the “U”, “V”, or “C” in the first position is smaller than in the second position.

According to at least one embodiment, the connection arm has an adaptable length in order to adapt to different positions which the radiation emitting units can assume relative to one another when moved, relative to each other.

According to at least one embodiment the connection arm comprises two end portions. According to at least one embodiment, at least one of the end portions is moveable with respect to the other one of the end portions, for example so as to adapt, e.g. increase or decrease, the length of the connection arm to different positions which the radiation emitting units assume relative to one another when moved, relative to each other.

According to at least one embodiment, the connection arm is telescopically adaptable in length, e.g. one or both of the two end portions of the connection arm can telescopically extend from or retract towards the connection arm. By extending telescopically the distance between the two end portions increases as does the length of the connection arm.

According to at least one embodiment the connection arm comprises a fully extended position, i.e. a position in which the end portions of the connection arm are at their furthest distance from each other and a fully retracted position, i.e. a position in which the end portions of the connection arm are closest to each other.

According to at least one embodiment, at least one radiation emitting unit, e.g. only one, is arranged between the at least two radiation emitting units to which the connection arm is connected.

According to at least one embodiment, the at least one radiation emitting unit is connected to the two radiation emitting units which are connected to the connection arm. The at least three radiation emitting units form a panel or arrangement of radiation emitting units. The panel or arrangement of radiation emitting units is hence connected via two radiation emitting units to the connection arm.

According to at least one embodiment, a panel or arrangement of radiation emitting units comprises five radiation emitting units, the panel or the arrangement being attached to the connection arm through two radiation emitting units.

According to at least one embodiment the radiation emitting units in the panel or arrangement of radiation emitting units are moveable with respect to one another. In particular the radiation emitting units, can be moved relative to one another in order to adjust the illumination device for irradiating a surface of a non-plane shape, where the shape of different surfaces to be illuminated may vary.

For example, the illumination device can be adjusted for irradiating a surface of cylindrical shape or a human head, which may be idealized or approximated by a cylinder. The radiation emitting units can then be arranged such that the radiation output areas of the radiation emitting units have all the same distance to a lateral surface of the cylinder defining the cylindrical shape.

According to at least one embodiment, the radiation emitting units may be arranged in a first irradiation position in which the radiation output areas of the radiation emitting units have all the same distance to a lateral surface of the cylinder defining a first cylindrical shape.

According to at least one embodiment, the radiation emitting units may be arranged in a second irradiation position in which the radiation output areas of the radiation emitting units have all the same distance to a lateral surface of the cylinder defining a second cylindrical shape, wherein the second cylindrical shape has a greater diameter than the first cylindrical shape.

According to at least one embodiment, the radiation emitting units can be arranged in a C-shape configuration and/or a semi-circle configuration. Particularly, they may all emit radiation onto the irradiation object, e.g. the human head, when in the C-shape or semi-circle configuration. The radiation of the radiation emitting units may overlap at the object location. In the C-shape configuration or semi-circle configuration, respectively, the angle between two adjacent radiation emitting units may be at least 100° or at least 110° and/or at most 150° or at most 130°, e.g. 120°. Such a configuration may result in a more homogeneous illumination, e.g. of a human face.

The angle between two radiation emitting units is, in particular, defined as the angle between the output areas and/or the radiation source carriers of the two radiation emitting units. The angle may be the smaller angle defined by the (plane) emission surfaces of the two radiation emitting units.

According to at least one embodiment, the radiation emitting units may be arranged such that the radiation output areas of the radiation emitting units are parallelly aligned along a plane. This would guarantee the possibility of irradiation a planar irradiation object such as a leg of a patient. According to at least one embodiment the parallel aligned position of the radiation emitting unit along a plane corresponds to the fully extended position of the connection arm.

According to at least one embodiment, the radiation emitting units can be arranged in, e.g. moved into, a linear arrangement. The linear arrangement may be particularly suitable for irradiating a leg or an arm.

According to at least on embodiment, the duration of the entire illumination session may vary depending on if the radiation emitting units are arranged such that the radiation output areas of the radiation emitting units are parallelly aligned along a plane or if the radiation emitting units are arranged in a C-shape configuration and/or a semi-circle configuration.

According to at least one embodiment the duration of the illumination session when the radiation emitting units are arranged in a C-shape configuration and/or a semi-circle configuration may be less than when the radiation emitting units are arranged such that the radiation output areas of the radiation emitting units are parallelly aligned along a plane.

According to at least one embodiment, in a C-shape configuration and/or a semi-circle configuration the duration of the illumination session may be less than or equal to one of the following values: 18 min, 17 min, 16 min, 15 min, 14 min, 13 min. Additionally or alternatively, the duration of the entire illumination session is greater than or equal to one of the following values: 10 min, 11 min, 12 min, 13 min. The duration of the illumination session may be for example 18 minutes.

According to at least one embodiment, when the radiation emitting units are arranged such that the radiation output areas of the radiation emitting units are parallelly aligned along a plane, the duration of the illumination session may be less than or equal to one of the following values: 25 min, 24 min, 23 min, 22 min, 21 min, 20 min, 19 min, 18 min, 17 min, 16 min, 15 min, 14 min, 13 min. Session durations up to 25 minutes, e.g. 22 minutes, are usually accepted by users. Additionally or alternatively, the duration of the entire illumination session is greater than or equal to one of the following values: 10 min, 11 min, 12 min, 13 min. The duration of the session may be between 10 min and 25 min, for example 22 minutes.

According to at least one embodiment, the radiation emitting units are tiltable relative to the connection arm. The radiation emitting units (e, g. the entire arrangement of the different radiation emitting units) may be tiltable along an axis which is oblique, e.g. perpendicular, relative to the axis along which interconnected radiation emitting units are movable, e.g. pivotable, relative to one another.

The radiation emitting units being tiltable offers a further degree of flexibility in the movability of the illumination device and, in particular, of the radiation emitting units such as to guarantee the possibility of irradiating different parts of an irradiation object at different angles and/or having different angles.

According to at least one embodiment, the radiation emitting units may be movable, e.g. tiltable, during an irradiation process in an illumination session.

The radiation emitting units may be connected via a joint connection to the connection arm, such as to permit the tilting movement of the radiation emitting unit. The joint connection may be any one of a hinge joint, saddle joint, pivot joint, ball and socket joint or combination thereof. Such joint connections increase the degrees of freedom in mobility of the radiation emitting units and as such guarantee a better positioning of the radiation emitting units with respect to an irradiation object.

The movement of the radiation emitting units may be controlled by the electronic control unit, for example depending on data values obtained by different sensors, e.g. a temperature sensor, radiation sensors, and/or distance sensors.

According to at least one embodiment, one or more radiation emitting units may be provided with handles.

The handles are particularly useful when moving the radiation emitting unit(s) manually to their intended position or desired configuration (e.g. C-shaped or linear arrangement) and/or for translating the panel or arrangement of radiation emitting units to a different position relative to the irradiation object.

According to at least one embodiment, the respective radiation emitting unit may have two opposite end surfaces connected by two opposite side surfaces. The respective unit may be elongate. In other words, the side surfaces may be longer than the end surfaces. The side surfaces may be parallel and/or the end surfaces may be parallel. The side surfaces may be oriented parallel to a pivot axis about which one radiation emitting unit is pivotable relative to the adjacent and connected unit.

According to at least one embodiment, the handles may be disposed at different locations on two radiation emitting units, e.g. at the respective back side of the radiation emitting unit. One handle may extend along a side surface at one unit and another handle may extend along an end surface at another unit.

According to at least one embodiment, two sections of the connection arm which may be movable relative to one another are provided with a respective handle. These sections may for example be the two end portions.

With the handle disposed at different sections of the connection arm, in particular on the respective end portions, the adaptation of length of the connection arm becomes manually easily possible and the connection arm can be brought, for example, in its fully extended or fully retracted position.

With the handle positioned on two different radiation emitting unit it becomes easier to tilt the radiation emitting units, e.g. the panel of radiation emitting units, such that the radiation output regions coincides with an irradiation object.

According to at least one embodiment, the connection arm is movably connected to a positioning arm of the illumination device. The connection may permit rotational movement, e.g. only rotational movement, of the connection arm relative to the positioning arm.

According to at least one embodiment the positioning arm is movably connected to the common support. The connection may permit rotational and/or pivoting movement, e.g. only rotational and/or pivoting movement, of the positioning arm relative to the common support.

The positioning arm may for example be used to set the connection arm and in particular the radiation emitting units at a predetermined and/or set distance from the irradiation object.

According to a further embodiment, the positioning arm is connected or connectable to the connection arm on a first end and to the common support on a second end.

According to at least one embodiment the positioning arm extends along a first axis and comprises a first and second end. The first end connects or is connectable to the connection arm and as such to the electromagnetic radiation emitting units. The second end is connected or is connectable to the common support, preferably to a top portion, e.g. a portion on the distal end of the common support with respect to the base/foot element(s)) of the common support.

The positioning arm may extend from the common support at an angle with respect to the longitudinal axis of the common support. The angle may be any angle between 1° to 179° but may lie in the range of 70° to 110°. The positioning arm may for example be pivotally connected to the common support such as to be positioned at different angles with respect to the longitudinal axis of the common support.

The positioning arm may extend substantially perpendicular to the connection arm.

According to at least one embodiment, the connection arm is moveably connected to the positioning arm via an attachment element extending along a longitudinal axis.

According to at least one embodiment, the attachment element may comprise connecting means for connecting to the positioning arm and/or to the connection arm. According to at least one embodiment the connecting means comprise a joint connection, e.g. a hinge joint, a saddle joint, a pivot joint, a ball joint or similar.

The connection arm may be connected to the positioning arm via the attachment element, such as to permit a rotation, e.g. of 180° or even 360° or more than 360°, with respect to the positioning arm.

According to at least on embodiment, the positioning arm is a spring arm, e.g. a gas-spring arm.

According to at least one embodiment, the illumination device comprises a gas spring, which is operatively connected to the positioning arm to support the positioning arm.

According to at least one embodiment the gas spring may have an extension force being less or equal to 4100 N, 4000 N, 3900 N, 3800 N, 3700 N. According to at least one embodiment the gas spring may have an extension force being more or equal to 3400 N, 3500 N, 3600 N, 3700 N.

Such an extension force is advantageous for holding the total weight of the support component and of the radiation emitting units without losing stability.

According to at least one embodiment the gas spring may have a weight less or equal than 1.5 kg, 1.4 kg, 1.3 kg, 1.2 kg. According to at least one embodiment the gas spring may have a weight being greater or equal than 1.1 kg, 1.2 kg, 1.3 kg. The gas spring may have for example a weight of 1.305 kg.

According to at least one embodiment, the illumination device comprises an irradiation object cooling system, e.g. a system to deliver cooling gas to the irradiation object.

The irradiation object may be for example a mammal suffering from a skin disease, e.g. a human patient suffering from a skin disease.

One problem related to a PDT is the amount of pain felt and endured by an irradiation object, e.g. the patient during an irradiation process. The pain felt by a patient frequently ranges from moderate to severe and has been recognized often as a reason for reluctancy towards a PDT treatment.

The pain can be caused by the topically applied substance performing its function and/or by the temperature increase on the skin surface caused by the radiation. The usual skin surface temperature of a patient, not being in contact with any heat source, is at ca. 32 °C. For further information regarding heat sensitivity of the skin of a person, reference is made to the work of Jeon & Caterina (2018): Chapter 4-Molecular basis of peripheral innocuous warmth sensitivity; Handbook of Clinical Neurology Volume 156, 2018, Pages 69-82 (https://doi.org/10.1016/B978-0-444-63912-7.00004-7).

During an illumination session, the temperature of the irradiation region of the irradiation object can increase to up to 40 °C. Increased temperatures, e.g. 42 °C, may cause a burning feeling on the skin of the patient, rendering the whole treatment unpleasant and decreasing the probability of a patient to repeat such a treatment although medically recommendable, especially since the photodynamic reaction involving reactive oxygen may enhance or amplify heat sensation or sensitize the skin to such heat sensation.

The irradiation object cooling system may provide some relief to the irradiation object.

According to at least one embodiment, the irradiation object cooling system comprises at least one cooling gas outlet which is configured to face towards the object location wherein the illumination device is configured such that cooling gas can leave the illumination device through the at least one cooling gas outlet.

According to at least one embodiment, the illumination device comprises a cooling gas driver system comprising at least one cooling gas driver, wherein the cooling gas driver system is configured to drive a cooling gas flow through the cooling gas outlet. The cooling gas driver may be comprised in a radiation emitting unit.

According to at least one embodiment, the cooling gas driver is a fan.

According to at least one embodiment, each radiation emitting unit comprises one or more cooling gas drivers.

According to at least one embodiment, at least one radiation emitting unit comprises at least one cooling gas outlet, which faces towards the object location.

According to at least one embodiment, at least one radiation emitting unit may comprise more than one cooling gas outlet. In particular, at least one radiation emitting unit may comprise one or two cooling gas outlets. For example, each radiation emitting unit may comprise one or two cooling gas outlets.

According to at least one embodiment, at least one radiation emitting unit comprises at least two cooling gas outlets, which, preferably, are disposed in opposite end regions of the radiation emitting unit, e.g. separated along the main longitudinal or an axis perpendicular to the main longitudinal axis of the radiation emitting unit.

With at least two cooling gas outlets arranged on opposing ends of a radiation emitting unit it is possible to cool an irradiation region of an irradiation object from different sides and/or angles and/or locations. If the irradiation object is a patient and the irradiation region is a portion of the skin of the patient, it may achieve a more uniform cooling distribution on the skin portion and as such a more pleasant feeling for the patient or at least reduce the pain burden.

According to at least one embodiment, the radiation source carrier comprises one or more cooling gas passages for the cooling gas flow from one side of the radiation source carrier to the opposite side of the radiation source carrier.

The radiation source carrier may have a continuous surface on which a plurality of radiation sources is arranged. During operation the temperature of the radiation sources increases. It is therefore advantageous to provide a cooling mechanism for the radiation sources.

The cooling gas passages have the indirect function of cooling the radiation source carrier and thereby also indirectly the radiation sources.

According to at least one embodiment, each cooling gas passage forms one cooling gas outlet or each cooling gas passage is fluidly connected to at least one cooling gas outlet.

According to at least one embodiment, the cooling of the radiation source carrier and of the irradiation region of the irradiation object may be combined. The cooling gas flow provided by the cooling gas driver may pass along the radiation source and/or through the cooling gas passages. This may guide loss heat away from the radiation sources (in case of LEDs there may not be particularly much loss heat but heat may have an influence on the wavelength/temperature, which influence should be kept as low as possible). Through the cooling gas outlet, the cooling gas may travel towards the irradiation object to cool the irradiation object.

According to at least one embodiment, the cooling gas passage may form a cooling gas outlet. The passage for example may end into an open distal end defining the cooling gas outlet.

According to at least one embodiment, each cooling gas passage is fluidly connected to at least one cooling gas outlet such that the cooling gas flow flows firstly through the passage and afterwards through the cooling gas outlet.

According to at least one embodiment, the radiation source carrier forms a cooling gas barrier and/or is closed, e.g. without cooling gas passages defined in the radiation source carrier. In such an embodiment, the cooling gas may not have to travel through the carrier. It may, for example pass the carrier laterally.

According to at least one embodiment, one or more cooling gas passages for the cooling gas flow from one side of the radiation source carrier to the opposite side of the radiation source carrier are arranged adjacent to an edge laterally delimiting the radiation source carrier.

By being arranged on the lateral edge of the radiation source carrier, the cooling fluid passages are arranged more proximal to the cooling gas outlets. As such, the loss of temperature of the cooling gas flow is minimized due to its path towards the cooling gas outlet. Cooling gas passages through the radiation source carrier can then be avoided. Contemporaneously, by passing along the lateral edges of the radiation source carrier, the cooling gas passages still have a cooling effect on the carrier and hence on the radiation sources on the carrier.

According to at least one embodiment, the respective radiation emitting unit comprises at least one cooling gas inlet, e.g. at least one inlet per gas driver. The cooling gas inlet may overlap with the radiation source carrier when seen in plan view onto the cooling gas inlet.

According to at least one embodiment, the illumination device is configured such that the cooling gas flows from a side of the radiation source carrier remote from the cooling gas outlet towards the cooling gas outlet. The cooling gas inlet(s) may be disposed on the side of the radiation source carrier facing away from the cooling gas outlet. The cooling gas inlet(s) may be disposed on the side of the radiation emitting unit facing away from a radiation emission surface.

According to at least one embodiment, the temperature of the cooling gas at the cooling gas outlet is greater than the ambient temperature and less than or equal to the temperature at a radiation source or a radiation cooling body (e.g. a heat sink or a heat spreader) which is thermally connected to one or more radiation sources of the radiation emitting unit.

According to at least one embodiment, the illumination device comprises an active radiation source cooling system. The active radiation source cooling system actively cools the radiation source, in particular during their operation in an irradiation process.

According to at least one embodiment, the radiation source cooling system comprises a gas driver, e.g. a fan, configured to move radiation source cooling gas relative to the radiation sources. The radiation source cooling system may comprise more than one gas driver, e.g. fans.

The gas driver of the radiation source cooling system may be the cooling gas driver described above but does not have to be, e.g. as separate cooling gas driver(s) are provided or as the illumination system does not have an irradiation object cooling system. The gas driver may move radiation sources cooling gas towards, through or in the proximity of one or more radiation sources to actively cool the radiation sources.

According to at least one embodiment, the active cooling system comprises one or more gas drivers, e.g. fans. The gas inlets may be positioned as the cooling gas inlets discussed above.

According to at least one embodiment, the illumination device is configured such that the radiation source cooling gas is used as cooling gas for cooling the irradiation object with the irradiation object cooling system. As such, only one cooling system is required, which functions for both the radiation sources and the irradiation object. This makes the illumination device lighter and easier to operate.

Next, a method for treating a skin disease is specified. The illumination device specified herein is expediently used for this method. All features disclosed in connection with the illumination device are therefore also disclosed for the method and vice versa.

According to at least one embodiment, the method comprises a step a), in which a pharmaceutical substance is applied to the surface of the skin in a region which is to be treated. In a step b), the skin region to be treated is arranged in a predetermined object location of an illumination device, e.g. the device according to any of the embodiments described herein. In a step c), the skin region to be treated is irradiated with the illumination device. In this step, the illumination session is performed.

According to at least one embodiment the method comprises the step of adjusting the operation of the illumination device, e.g. adjusting the radiation emitted by the radiation sources, on the basis of temperature dependent variation in wavelength of the emitting radiation and/or on the basis of temperature dependent variations in the optical output power.

According to at least one embodiment the method comprises the step of cooling the skin region to be treated with the irradiation object cooling system.

The skin disease or disorder may be or may comprise a neoplastic skin disease, like actinic keratosis, basal cell carcinoma, squamous cell carcinoma in situ, warts, acne, wound healing disorders/chronic wounds, bacterial and/or fungal infections or inflammatory skin diseases. For example, the pharmaceutical substance is suitable to be topically applied to the skin in a region to be treated. It should be noted that the present disclosure covers therapeutic and non-therapeutic methods.

According to at least one embodiment, the pharmaceutical substance is a photosensitizing drug or precursor to such a drug that is excitable by light in the radiation spectrum emitted by the illumination device.

According to at least one embodiment, the pharmaceutical substance comprises 5-aminolevulinic acid. 5-aminolevulinic acid has been well studied and is considered a reliable prodrug for generating a photosensitizer.

According to at least one embodiment, the skin disease is or comprises a neoplastic skin disease like actinic keratosis, basal cell carcinoma, squamous cell carcinoma in situ, or warts, acne, wound healing disorders/chronic wounds, bacterial and/or fungal infections, inflammatory skin diseases.

Next, a method for operating an illumination device is specified. Particularly, an illumination device specified herein can be operated with this method. All features disclosed in connection with the illumination device are therefore also disclosed for the method and vice versa.

According to at least one embodiment, the method comprises a step in which a measurement signal is provided, said measurement signal being indicative for a distance between the radiation emitting unit and the irradiation object. In a further step, an operation signal is generated as a function, i.e. depending on, of the measurement signal, said operation signal being configured to cause the illumination device to adjust the operation of the illumination device or to call for an adjustment of the operation of the illumination device.

According to at least one embodiment, the method comprises a step in which a measurement signal is provided, said measurement signal being indicative for temperature dependent variations in the wavelength of the radiation emitted and/or for temperature dependent variations in the optical output power.

In a further step, an operation signal is generated as a function, i.e. depending on, of the measurement signal, said operation signal being configured to cause the illumination device to adjust the wavelength of the radiation emitted by the radiation sources of the illumination device or to call for an adjustment of the wavelength of the radiation emitted by the radiation sources of the illumination device, e.g. by the user such as a medical practitioner.

Additionally, or alternatively in a further step an operation signal is generated as a function, i.e. depending on, of the measurement signal, said operation signal being configured to cause the illumination device to adjust the optical output power of the illumination device or to call for an adjustment of the optical output power of the illumination device, e.g. by the user such as a medical practitioner.

According to at least one embodiment, the method comprises a step in which a measurement signal is provided, said measurement signal being indicative for a temperature at the irradiation region of the irradiation object. In a further step, an operation signal is generated as a function, i.e. depending on, of the measurement signal, said operation signal being configured to cause the illumination device to adjust the operation of the irradiation object cooling system of the illumination device or to call for an adjustment of the operation of the irradiation object cooling system of the illumination device.

Furthermore, a computer program product is specified. The computer program product comprises machine-readable instructions, which, when loaded and executed on a processor, are configured to cause the illumination device to execute one of the embodiments of the method for operating the illumination device. The processor may be part of the illumination device.

Moreover, a computer-readable medium is specified, having stored thereon the computer program product. The medium may be a non-transitory storage medium.

### BRIEF DESCRIPTION OF THE DRAWINGS

The present disclosure is further illustrated by the following figures and examples, however, without being restricted thereto.

FIGS. 1 and 2 show a side view and a detailed view of an exemplary embodiment of an illumination device, respectively.

FIGS. 3 and 4 show a simplified schematic exemplary arrangement of the radiation emitting units.

FIG. 5 shows an exemplary embodiment of a radiation emitting unit.

FIG. 6 shows an exemplary embodiment of a mechanical connection system.

FIG. 7 shows another exemplary mechanical connection system with radiation emitting units connected therewith.

FIG. 8 shows further configurations of an exemplary mechanical connection system.

FIG. 9 shows further configurations of an exemplary mechanical connection system.

FIG. 10 shows a perspective view of an exemplary embodiment of a positioning arm.

FIGS. 11a, 11b show a single exemplary radiation emitting unit comprising an active radiation source cooling system.

FIG. 12 shows an exemplary embodiment of a method for treating a skin disease on the basis of a flow chart.

### DETAILED DESCRIPTION OF THE DRAWINGS

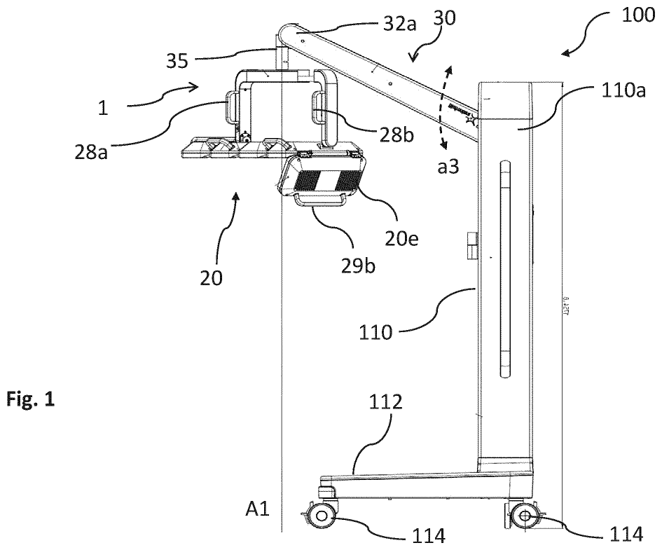

FIG. 1 shows a side view of an exemplary illumination device 100.

The illumination device 100 comprises a common support 110. The common support extends longitudinally along an axis A1 and comprises a foot element 112 comprising wheels 114, in order to be movable.

Positioning arm 30 is connected to a top end portion 110a of the common support 110 and extends angularly with respect axis A1.

The positioning arm 30 can pivot, i.e. swing, vertically with respect to the common support 110 element according to the arrow a3. A connection arm 1 is connected via an attachment element 35 to a first end 32a of the positioning arm 30.

The connection arm 1 further comprises a radiation emitting unit panel 20 connected to it, comprising five radiation emitting units 20, wherein one radiation emitting unit 20e is shown being tilted with respect to the panel 20.

The connection arm 1 further comprises two handles 28a and 28b on its two end portions. The radiation emitting units 20 also comprises four handles, one of which, e.g. handle 29b, is shown attached to the radiation emitting unit 20e.

The illumination device 100 may be configured to consider temperature dependent variations in the wavelength of the radiation emitted by the radiation sources (not shown) and/or temperature dependent variations in the optical output power, e.g. in the radiation power, for adjusting parameters of the illumination session in order to irradiate the predetermined radiation dose onto the irradiation region.

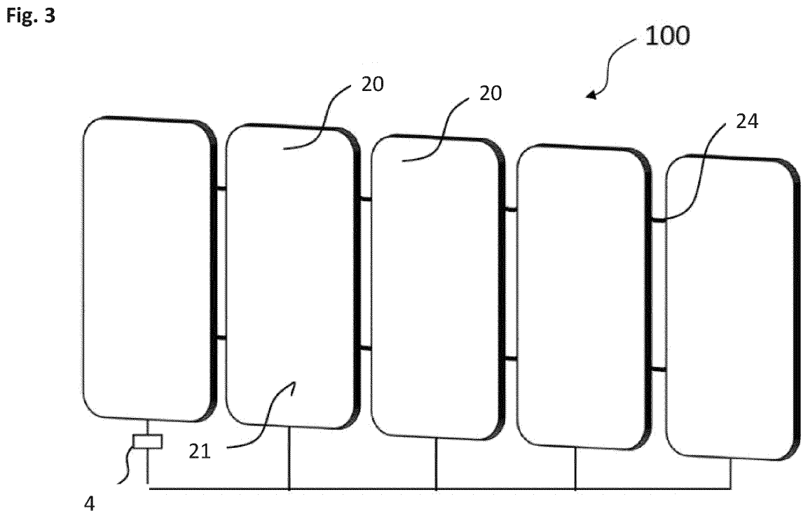

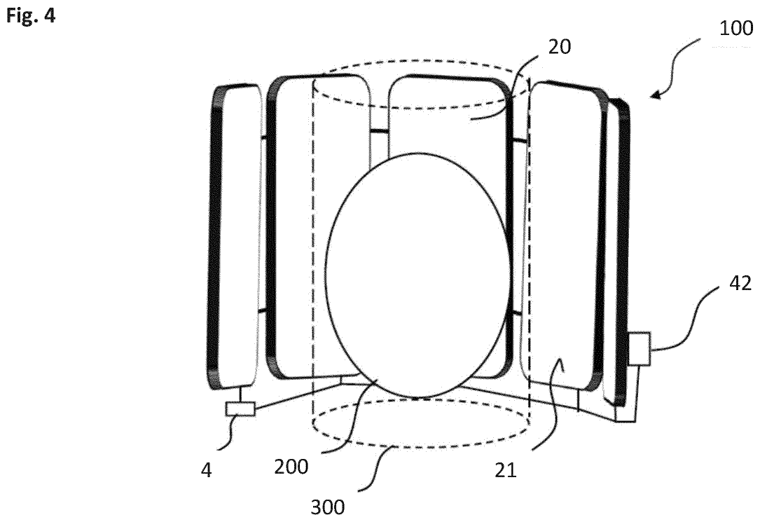

The operation of the illumination device 100 may adjusted by using one of, an arbitrary combination of or all of the following measures:
varying the distance between the respective radiation emitting unit 20 and the irradiation object (see e.g. FIGS. 3 and 4),
adjusting the radiation power emitted by the respective radiation emitting unit 20, and/or
adjusting a duration of the illumination session

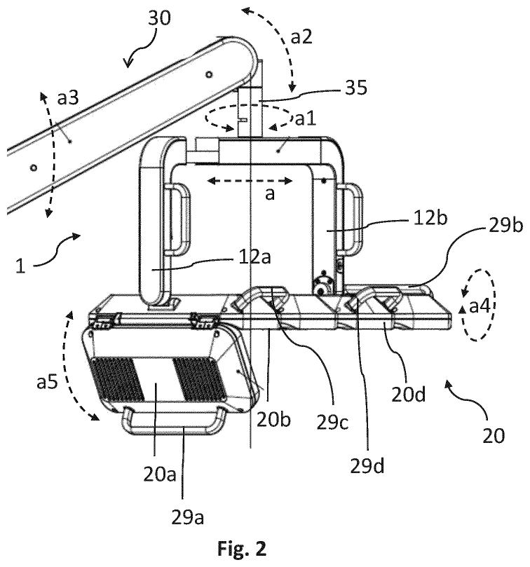

FIG. 2 shows a detailed view of the connection mechanism and movement possibilities of various elements of an illumination device, based on the exemplary embodiment of FIG. 1. Parts not described in this figure are explained in FIG. 1.

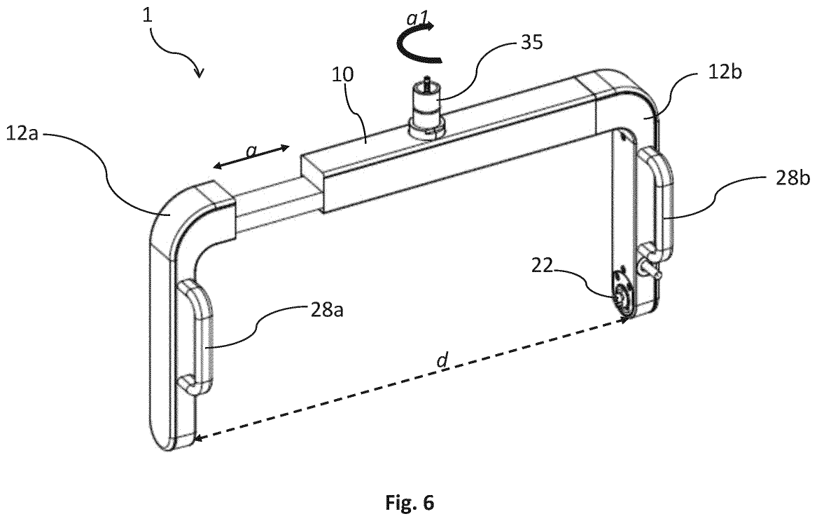

As will be explained in more detail in FIG. 6, the connection arm 1 can adapt its length. Specifically, one end portion 12a can extend as shown by arrow a, thereby increasing or decreasing the distance between the two end portions 12a, 12b. Also both end portions 12a, 12b may be moveable.

The connection arm 1 is connected to the positioning arm 30 via an attachment element 35. The attachment element 35 can rotate as shown by arrow a1, thereby rotating the connection arm 1 according to arrow a1.

Additionally, or alternative the connection arm 1 may be able to rotate with respect to attachment element 35 and according to arrow a1.

The attachment element 35 is also configured to be able to swing with respect to the positioning element 30 and according to arrow a2. The positioning element 30 can swing according to arrow a3 with respect to the common support 110 shown in FIG. 1.

The attachment element 35 may comprise connecting means (not shown) for connecting to the positioning arm 30 and/or to the connection arm 1. The connecting means may comprise a joint connection, e.g. a hinge joint, a saddle joint, a pivot joint, a ball joint or similar.

The connection arm 1 may be connected to the positioning arm 30 via the attachment element 35, such as to permit a rotation, e.g. of 180° or even 360° or more than 360°, with respect to the positioning arm 35 as indicated by arrow a1.

The panel 20 of radiation emitting units is adapted to tilt according to arrow a4 as explained in the following figures. Each radiation emitting unit 20a to 20e is capable to move with respect to the other radiation emitting for example by swinging, as shown by arrow a5.

The radiation emitting units of the panel of radiation emitting units 20 further comprise four handles, 29a, 29b, 29c, 29d. The handles 29a and 29b are arranged, respectively on the most external radiation emitting units 20a and 20e of the panel 20 and two handles 29c and 29d respectively on the radiation emitting units 20b and 20d which are directly connected to the connection arm 1.

The handles 29a and 29b are arranged on the most external side of the respective radiation emitting units relative to the panel 20. They are used e.g. to swing the respective radiation emitting unit, e.g. radiation emitting unit 20e relative to the other radiation mitting units 20b to 20e.

The handles 29c and 29d instead are arranged on a top or bottom portion of the radiation emitting units 20b and 20d. These handles 29c, 29d are used to move the whole connection arm 1 and/or panel 20.

FIG. 3 shows an exemplary schematic embodiment of a part of the illumination device 100 for photodynamic therapy. The part of the illumination device 100 comprises several radiation emitting units 20 which are linearly connected to each other. The radiation emitting units 20 are movably, especially pivotally, connected to each other. For this purpose, hinges 15 are used between the radiation emitting units 20. The radiation emitting units 20 each comprise a radiation output region 21 through which radiation generated by the respective radiation emitting unit 20 is coupled out of the illumination device 100. The output regions 21 are, for example, in each case formed by a (plexiglass or glass) cover plate of the respective radiation emitting unit 20.

In FIG. 3, the illumination device 100 is configured to irradiate a plane surface. The radiation emitting units 20 are arranged such that the radiation output regions 21 lie substantially in a common plane. Main radiation directions of the radiation emitting units 20 are substantially parallel to each other.

The illumination device 100 comprises an electronic control unit 4 which is configured to control the operation of the illumination device 100, wherein the respective radiation emitting units 20 are operatively coupled to the electronic control unit 4.

The illumination device 100 may further comprise at least one temperature sensor (not shown) that may be operatively connected to the electronic control unit 4 to provide temperature data to the electronic control unit 4. The electronic control unit 4 may be configured to adjust the operation of the illumination device 100 based on the temperature data to ensure that the predetermined radiation dose is delivered to the irradiation object.

The at least one temperature sensor may be located at or near a radiation source. In case of more radiation sources, a temperature sensor at each radiation source may be provided or just one or more temperature sensor(s) at one or more of the radiation sources, e.g. one temperature sensor per radiation emitting unit 20.

In case of more temperature sensors, each temperature sensor may be operatively connected to the electronic control unit 4 to provide temperature values. The electronic control unit 4 may average some or all of the different values such as to obtain a single temperature data value for some or all of the radiation sources.

The at least one temperature sensor may continuously provide temperature data during operation of the illumination device. The temperature sensor may be polled with an appropriate frequency The illumination device 100 may further comprise a radiation sensor (not shown) which is arranged to receive radiation emitted from the illumination device 100 in order to generate radiation data which is characteristic for a wavelength shift of the peak wavelength of the radiation source, wherein the electronic control unit 4 may be configured to adjust the operation of the illumination device 100 based on the radiation data to ensure that the predetermined radiation dose is delivered to the irradiation region.

The radiation sensor and/or the temperature sensor may be physically connected to the electronic control unit 4, e.g. through a cable connection, or wirelessly operatively connected, e.g. via Bluetooth, via Wi-Fi or similar.

The radiation sensor may continuously provide radiation data during operation of the illumination device 100. The radiation sensor may be polled with an appropriate frequency.

One or more radiation sensors may be located at or near a radiation source and may be configured to receive the radiation reflected from an irradiation object. The data is then provided to the electronic control unit 4 which based on the received information may extrapolate how much radiation has been absorbed by the irradiation object and may adjust the radiation dose accordingly.

A radiation sensor may provided for each radiation emitting unit 20. Each radiation sensor may then be configured to receive the radiation reflected from an irradiation object to its respective radiation emitting unit.

FIG. 4 shows a further schematic view of the illumination device 100 of FIG. 3 in a different configuration, in which the illumination device 100 is configured to irradiate a surface of non-plane shape, namely a cylinder surface, particularly a human face. The radiation emitting units 20 are arranged in a C-shape configuration. For this purpose, the radiation emitting units 20 have been pivoted relative to each other so that the distances of the radiation output regions 21 of the radiation emitting units to the cylinder surface are substantially the same. The rearrangement or movement of the radiation emitting units 20 can be done manually. In the present case, each radiation emitting unit 20 is assigned a motor 42, which is configured to move/pivot the respective radiation emitting unit 20 relative to the further radiation emitting units 20.

The radiation emitting units may however also bee moveable manually, for example through aid of the handles described in relation to FIGS. 1 and 2.

The cylinder around which the radiation emitting units 20 are arranged defines a predetermined object location 300. The object location 300 is arranged at a distance to the radiation output regions 21 of the radiation emitting units 10. Inside the object location 300, an irradiation object 200 is arranged. The irradiation object 200 is, for example, a human head. The head 200 is therapeutically treated by irradiating with the illumination device 100.

The duration of the entire illumination session may vary depending on if the radiation emitting units 20 are arranged such that the radiation output areas of the radiation emitting units 20 are parallelly aligned along a plane or if the radiation emitting units are arranged in a C-shape configuration and/or a semi-circle configuration. In the C-shape configuration it may last less than when the radiation emitting units 20 are arranged such that the radiation output areas of the radiation emitting units 20 are parallelly aligned along a plane, for example 18 minutes compared to 22 minutes.

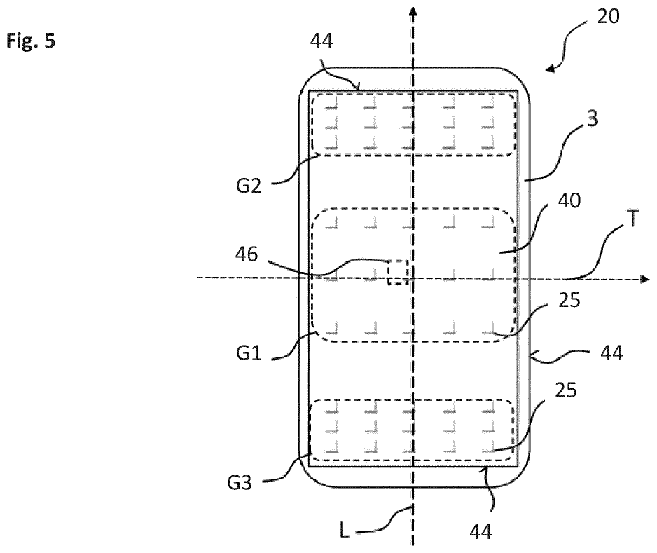

FIG. 5 shows an exemplary embodiment of a radiation emitting unit 20 in plan view of the radiation emitting unit 20, for example in plan view of the cover plate. The radiation emitting unit 20 of FIG. 5 is, for example, used for all radiation emitting units 20 in the illumination device 100 of FIGS. 1 to 4.

The radiation emitting unit 20 comprises a unit housing 3, for example comprising metal and/or plastic, and a radiation source carrier 40 laterally surrounded by the unit housing 3 in the shown plan view. The unit housing 3 defines a lateral edge 44 of the radiation emitting unit 20 delimiting the radiation emitting unit 20 in a transversal direction T.

The radiation source carrier 40 is, for example, a printed circuit board, PCB for short. The radiation source carrier 40 is an elongated, rectangular shaped carrier. A main direction of extension of the radiation source carrier 40 defines a longitudinal direction L. A direction perpendicular to the longitudinal direction L and running parallel to a main extension plane of the radiation source carrier 40 defines the transversal direction T. The radiation source carrier 40 is delimited in the longitudinal direction L and in the transverse direction T by carrier edges 42.

A plurality of radiation sources 25 is arranged on the radiation source carrier 40. The exact positions of the radiation sources 25 on the radiation source carrier 40 is indicted by the intersection points of the squared brackets. For example, a center of a chip surface of a semiconductor chip assigned to the radiation source overlaps with the respective intersection point.

In the exemplary embodiment, all radiation sources 25 of the radiation emitting unit 20 are arranged on a common radiation source carrier 40. During intended operation, all radiation sources 25 preferably emit radiation in the visible spectrum and of essentially the same color and/or with essentially the same peak wavelength.

The emission spectrum of the radiation sources 25 may have a peak wavelength in one of the following ranges: 634 nm±5 nm, 635 nm±5 nm, 542 nm±5 nm, 506 nm±5 nm, 417 nm±5 nm, 420 nm±5 nm. Particularly, this peak wavelength may be obtained at an operating temperature of the optoelectronic component below 50 °C, for example at 25 °C, and at operating currents between 100 mA and 1000 mA, inclusive. The half-band width of the spectrum may be, e.g. at least 10 nm and/or at most 20 nm, e.g. 16 nm.

The radiation sources 25 may further emit radiation of the same or similar peak wavelengths, e.g. radiation of the same color, e.g. red light (635 nm±4 nm), blue light (420 nm±4 nm), yellow light (542 nm±4 nm) or green light (506 nm±4 nm).

An entire illumination session may have a duration less than or equal to one of the following values: 20 min, 19 min, 18 min, 17 min, 16 min, 15 min, 14 min, 13 min. Session durations up to 20 minutes are usually accepted by users. The duration of the entire illumination session may be greater than or equal to one of the following values: 10 min, 11 min, 12 min, 13 min. The duration of the session may be between 10 min and 20 min, for example, e.g. 18 minutes.

As can be seen in FIG. 5, the radiation sources 25 are arranged on the carrier 40 in three different groups G1, G2, G3, wherein each radiation source 25 is uniquely assigned to one group G1, G2, G3. The groups G1, G2, G3 are indicated by the dashed rectangles. A first group G1 with 15 radiation sources 25 is located in a center region of the radiation source carrier 40. A second G2 and a third G3 group, each with 15 radiation sources 25, are located on peripheral regions of the radiation source carrier 40. When viewed along the longitudinal direction L, the second G2 and third G3 group are located before and behind the first group G1. Within each group G1, G2, G3 the radiation sources 25 are arranged in a two-dimension regular group pattern. The group patterns of the second G2 and third G3 group are identical, whereas the group pattern of the first group G1 is different.

In the second G2 and the third G3 group, the radiation sources 25 are arranged more densely on the radiation source carrier 40 than in the first group G1. Thus, the occupancy density of the radiation source carrier 40 with radiation sources 25 in the second G2 and third group G3 is greater than in the first group G1. This arrangement is particular advantageous in terms of a homogeneous irradiation of the irradiation object along the longitudinal direction L.

As can also be seen in FIG. 5, the distance between two adjacent groups G1, G2, G3 is greater than a distance between the radiation sources 25 within a group G1, G2, G3 (the distance between two adjacent groups is the shortest distance between two radiation sources 25 of these two groups). Moreover, it is visible form FIG. 5 that the two-dimensional pattern in which the radiation sources 25 are arranged on the radiation source carrier 40 is symmetric with respect to an axis running parallel to the longitudinal direction L and also with respect to an axis running parallel to the transversal direction T.

In the exemplary embodiment of FIG. 5, a radiation source 25 is arranged in the geometric center of the radiation source carrier 40. Slightly offset from this geometric center, a distance sensor 46 is arrange on the radiation source carrier 2. The distance sensor 46 is, for example, a time-of-flight sensor comprising a laser diode. A distance to the adjacent radiation source 25 in the geometric center is, e.g., 10 mm.

Additionally or alternatively, the distance sensor 46 may be slightly offset, e.g. by at least 5 mm and at most 40 mm, form the center of a radiation field created by the radiation sources of the radiation emitting unit. The center of the radiation field may be the position of the center of mass when integrating over all the radiation sources of the radiation emitting unit.

FIG. 6 shows an exemplary back view of a mechanical connection mechanism comprising a connection arm 1 without the at least two radiation emitting units. The connection arm 1 has a U-shape. The connection arm 1 may however also be V-or C-shaped.

The connection arm 1 comprises a main portion 10 and two end portions 12a, 12b. As can be seen from the double headed arrow a, the end portions 12a can be moved along a longitudinal axis defined by the main portion 10 such as to increase the distance d between the two end portions 12a, 12b. As such the length of the connection arm 1 can be adapted.

The main portion 10 of the connection arm 12 comprises an attachment element 35, for attaching the connection arm to the positioning arm (see e.g. FIG. 1). The main portion 10 and as such the connecting arm 1 may be rotated with respect to the attachment element 35 as denoted by the arrow a1.

The end portions 12a, 12b also comprise connection means 22 (only shown for end portion 12b) for connecting respective radiation emitting units, as will be shown in the next figure.

The connection arm 1 further comprises two handles 28a, 28b, which in this example are attached on the end portions 12a, 12b of the connection arm 1.

The two end portions 12a, 12b of the connection arm may however also be independently telescopically extendable or retractable.

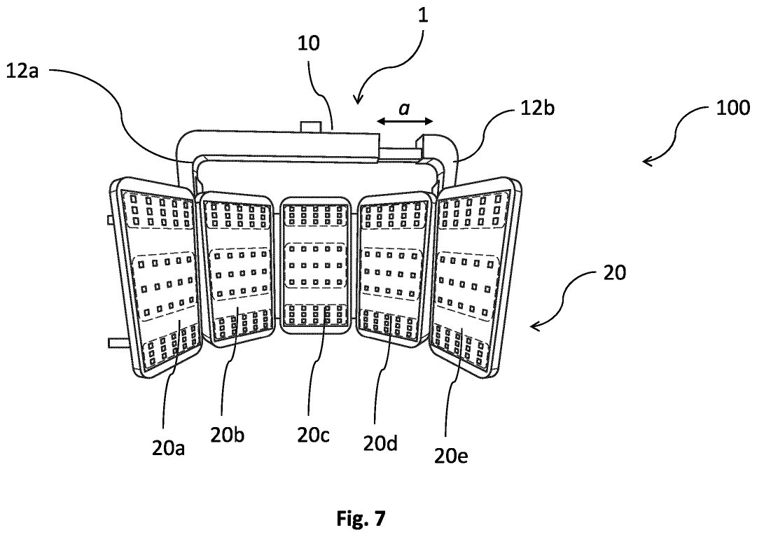

FIG. 7 shows an exemplary mechanical connection system comprising for example the connection arm 1 of FIG. 6 and a panel 20 of five radiation emitting units 20a to 20e rigidly axially connected to the connection arm 1, e.g. connected to the end portions 12a, 12b of the connection arm 1. The radiation emitting units 20a to 20b may be releasable connected to each other and/or to the connection arm.

The radiation emitting units 20b and 20d are connected to the end portion 12a and 12b of the connection arm 1, respectively. One radiation emitting unit 20c is arranged between the two radiation emitting units 20b and 20c. A radiation emitting unit 20a and 20e is respectively arranged at a side of the radiation emitting unit 20b and 20d, such as to form a panel or an arrangement 20.

The radiation emitting units 20a to 20e of the panel 20 are movable relative to one another and connected with one another. The radiation emitting units 20a to 20e are connected to each other through hinges in the back of the radiation emitting units (see for example FIG. 3).

Each radiation emitting unit 20a to 20e may comprise an own unit housing 3 (shown in FIG. 5) and may be an own housed module.

The connection arm 1 can adapt its length, as shown by the arrow a, in order to adapt to different positions which, the radiation emitting units 20a to 20e assume relative to one another when moved, e.g. pivoted, relative to each other. By extending the end portion 12a the wideness of the U-shaped panel 20 can change.

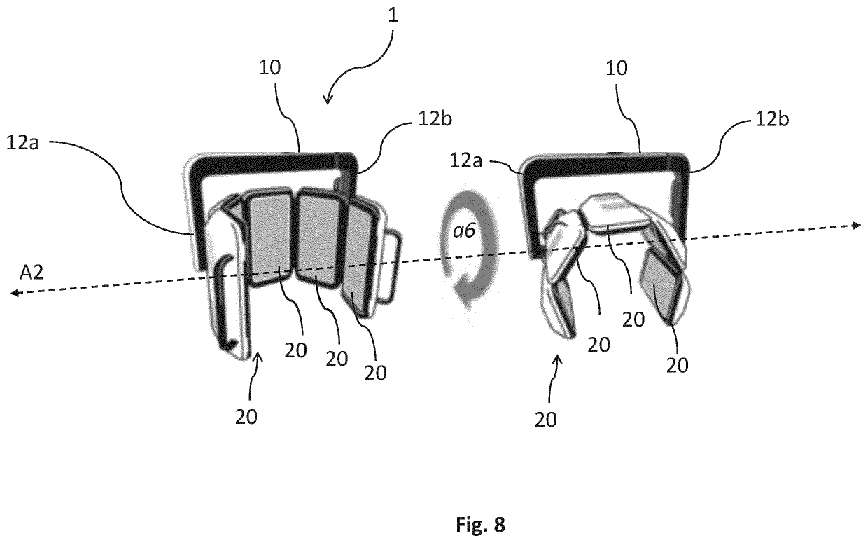

FIG. 8 shows a configuration of an mechanical connection system, e.g. the mechanical connection system of FIG. 7, in which the tiltability of the radiation emitting units 20 is shown.

The panel 20 of radiation emitting units 20 is tiltable, e.g. rotatable with respect to an axis A1 connecting the two distal ends of the connection arm and extending parallel to the longitudinal main axis of the main portion 10 of the connection arm. In other words, the radiation emitting units 20 are tiltable relative to the connection arm 1 along an axis which is oblique, e.g. perpendicular, relative to the axis along which the interconnected radiation emitting units 20 are movable, e.g. pivotable, relative to one another. In certain embodiments the radiation emitting units 20 can rotate up to 360° with respect to the axis A2 as shown by arrow a6.

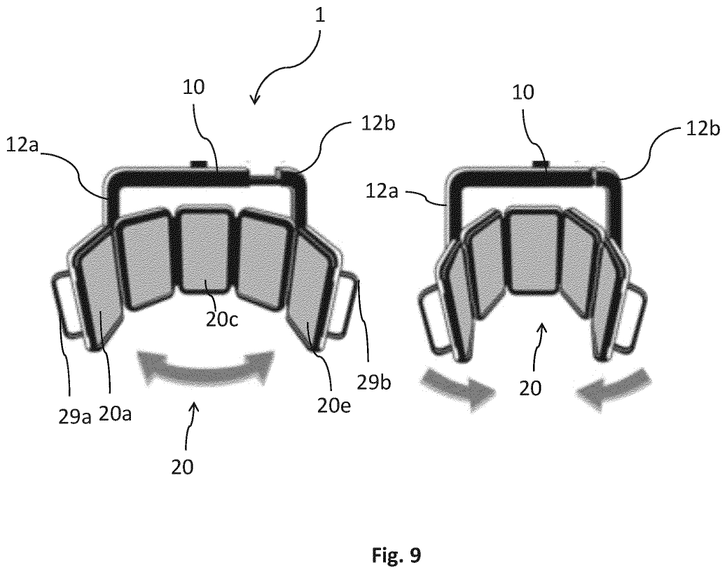

FIG. 9 shows a configuration of an mechanical connection system, e.g. the mechanical connection system of FIG. 7, in which the length adaptation of the connection arm 10 is shown.

As can be seen, the connection arm may adapt its length through the movability of the end portions 12a, 12b. In this example however only the movability of end portion 12b is moveable. By extending one or both of the distal ends 12a, 12b the wideness of the U-shaped panel 20 can change.

Specifically, by extending one or both end portions 12a, 12b along the longitudinal axis of the main portion 10 of the connection arm 1, the wideness of the radiation emitting units panel 20 changes (shown by the arrows) thereby permitting the irradiation of bigger or smaller irradiation objects and/or thereby changing the distance between the radiation emitting units and the irradiation object. The radiation emitting units 20 may also be parallelly aligned along a plane. Such a case would be for example beneficial in treating a leg or similar.

The more the distal end extends, the wider the U-shape of the panel. In a retracted position of the end portions 12a, 12b (figure on the right), the U-shape is at its narrowest.

The radiation emitting units 20a and 20e may further be moved independently relative to the other radiation emitting units through the handles 29a and 29b.

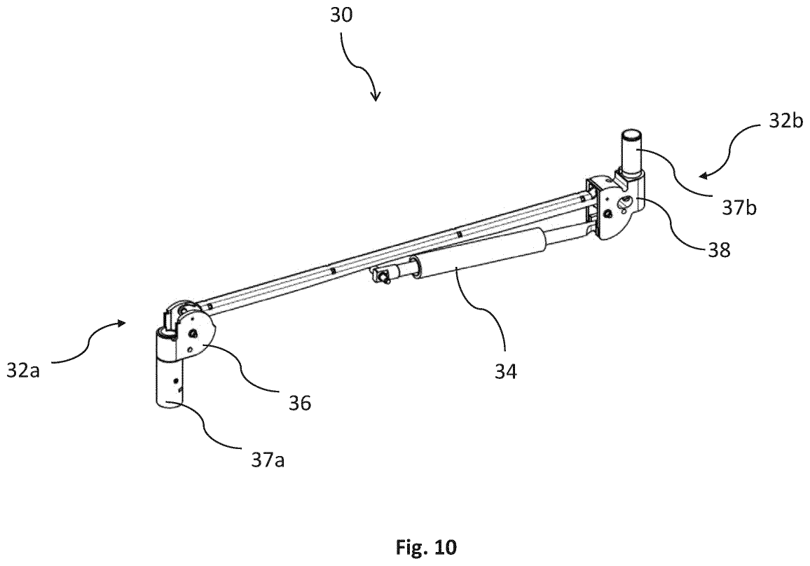

FIG. 10 shows a perspective view of an exemplary positioning arm 30, e.g. a spring arm 30.

The positioning arm 30 comprises a first end 32a and a second end 32b. The first end 32a comprises first connection means 36, which in this example is formed as a hinge joint. The connecting pipe 37a extending from the connection means 36 connects with the connection element of the connection arm, e.g. to the attachment element (not shown).

The second end 32b comprises second connection means 38, which in this example is formed as a hinge joint. The connecting pipe 37b extending from the connection means 38 connects with the common support of the illumination device (not shown).

The positioning arm 30 comprises a gas spring 34, connecting the second end 32b of the positioning arm 30 with a central part of the positioning arm 30. The gas spring 34 may have an extension force of 3900 N and a weight of 1,305 kg. The gas spring 34 supports the positioning arm 30 in holding the connection arm and the radiation emitting unit as will be shown in the next figure.

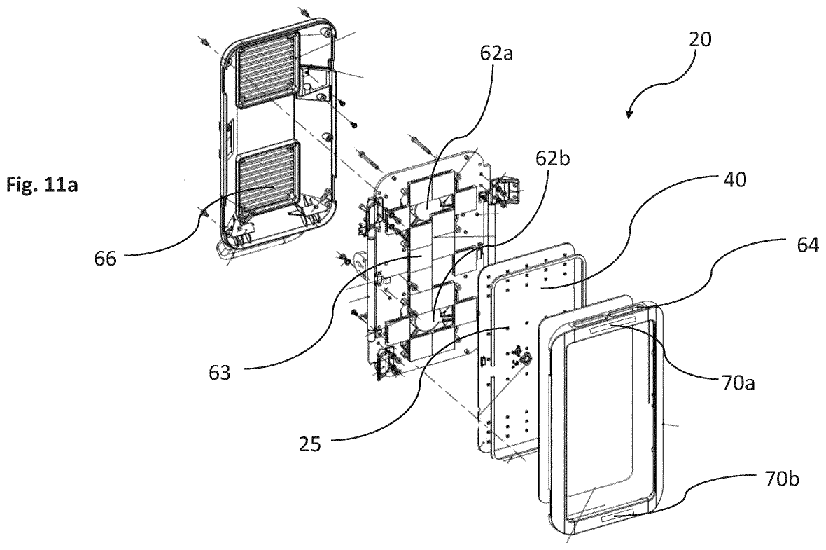

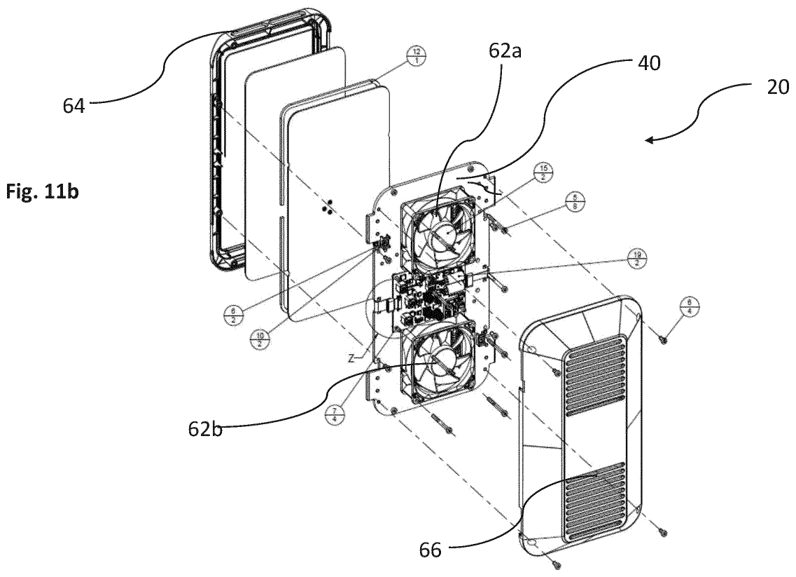

FIGS. 11a and 11b show an explosion view of a radiation emitting unit 20 seen from the front and from the back, respectively, comprising a radiation source cooling system.

As can be seen, the radiation emitting unit 20 comprises two cooling gas drivers 62a, 62b in order to cool the radiation source carrier 40 and in particular the radiation sources 25 (visible only in FIG. 11b) as well as several heat sinks 63 positioned towards the rear surface of the radiation source carrier 40.

The gas drivers 62a and 62b are in this cases fans configured to cool down the radiation source carrier 40, thereby cooling down the radiation sources 25. The rear surface of the radiation emitting units comprises ventilation grates 66 aligned with the gas driver e.g. the fans. The warm air exits the radiation emitting unit 20 from cooling gas outlets 64, e.g. ventilation slots 64, arranged e.g. at the top front of the radiation emitting unit 20.

The radiation emitting unit may further comprise an irradiation object cooling system comprising two cooling gas outlets 70a and 70b positioned in this example at the front surface on a top side and on a low side of the radiation emitting unit 20.

The cooling of the radiation source carrier 40 and of an irradiation region of an irradiation object, e.g. a patient, may be combined. The cooling gas flow provided by the cooling gas driver 62a and 62b may pass along the radiation sources 25 and/or through cooling gas passages (not shown). This may guide loss heat away from the radiation sources 25. Through the cooling gas outlets 70a and 70b, the cooling gas may travel towards the irradiation object to cool the irradiation object.

The temperature of the cooling gas at the cooling gas outlet may be greater than the ambient temperature and less than or equal to the temperature at a radiation source cooling body which is thermally connected to one or more radiation sources of the radiation emitting unit.

The radiation source carrier 40 may comprise one or more cooling gas passages (not shown) for the cooling gas flow from one side of the radiation source carrier 40 to the opposite side of the radiation source carrier, e.g. adjacent to an edge laterally delimiting the radiation source carrier 40. The cooling gas passage may form cooling gas outlets, e.g. ventilation slots 64 or cooling gas outlets 70a and 70b. The passage for example may end into an open distal end defining the cooling gas outlet 70a and 70b. Each cooling gas passage may be fluidly connected to at least one cooling gas outlet 70a and 70b, such that the cooling gas flow may flow firstly through the passage and afterwards through the cooling gas outlets 70a and 70b.

The radiation source carrier 40 may form a cooling gas barrier and/or may be closed, e.g. without cooling gas passages defined in the radiation source carrier 40. The cooling gas may therefore not have to travel through the carrier. It may, for example pass the carrier laterally.

The respective radiation emitting unit 20 may comprise at least one cooling gas inlet (not shown), e.g. at least one inlet per gas driver 62a and 62b. The cooling gas inlet may overlap with the radiation source carrier 40. The cooling gas inlet(s) may be disposed on the side of the radiation source carrier 40 facing away from the cooling gas outlet. The cooling gas inlet(s) may be disposed on the side of the radiation emitting unit 20 facing away from a radiation emission surface.

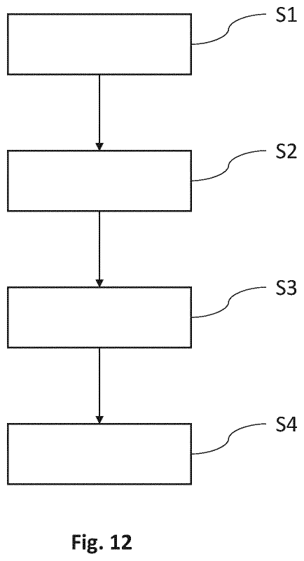

FIG. 12 shows an exemplary embodiment of the method for treating a skin disease on the basis of a flow chart. In a step S1, a pharmaceutical substance is applied to the surface of the skin of a human being in a region which is to be treated. Such a region might, for example, be a face portion of a human being, e.g. a patient. It might be the region in a face. The pharmaceutical substance is, for example, a photosensitizing drug or precursor to such a drug that is excitable by light in the radiation spectrum emitted by the illumination device 100. The pharmaceutical substance may comprise 5-aminolevulinic acid.

In a step S2, the skin region to be treated is arranged in the predetermined object location 300 of the illumination device 100 (see e.g. FIG. 3).

In a step S3, the skin region to be treated is irradiated with the illumination device, for example for at least 10 min and at most 20 min. During the illumination session, the skin region is irradiated with a predetermined radiation dose of, e.g., at least 30 J/cm² and at most 45 J/cm², such as 37 J/cm². A radiation dose of 37 J/cm² is especially suitable if red light with a wavelength of about 635 nm is used to irradiate the object. In case green or blue light is used, e.g. for irradiating a skin surface onto which ALA has been topically applied before the irradiation, the total radiation dose applied to the irradiation object during the illumination session may have a different value due to the different absorption properties for these wavelengths. The general teaching in the present disclosure does not only apply for light sources emitting red light but also for light sources of different colored light, e.g. blue or green light, particularly if ALA-based PDT is performed.

In a step S4, the radiation emitted by the radiation sources is adjusted on the basis of temperature dependent variation in wavelength of the radiation and/or the optical output power is adjusted on the basis of temperature dependent variations in the optical output power.

Step S4 may also comprises cooling the skin region to be treated with the irradiation object cooling system.

The skin disease or disorder may be or may comprise a neoplastic skin disease, like actinic keratosis, basal cell carcinoma, squamous cell carcinoma in situ, warts, acne, wound healing disorders/chronic wounds, bacterial and/or fungal infections or inflammatory skin diseases.

The pharmaceutical substance may be a photosensitizing drug or precursor to such a drug that is excitable by light in the radiation spectrum emitted by the illumination device.

The pharmaceutical substance may comprise 5-aminolevulinic acid. 5-aminolevulinic acid has been well studied and is considered a reliable prodrug for generating a photosensitizer.

The skin disease may be a neoplastic skin disease like actinic keratosis, basal cell carcinoma, squamous cell carcinoma in situ, or warts, acne, wound healing disorders/chronic wounds, bacterial and/or fungal infections, inflammatory skin diseases.

The invention described herein is not limited by the description in conjunction with the exemplary embodiments. Rather, the invention comprises any new feature as well as any combination of features, particularly including any combination of features in the patent claims, even if said feature or said combination per se is not explicitly stated in the patent claims or exemplary embodiments.

### REFERENCE NUMERALS

1 connection arm
3 unit housing
4 electronic control unit
10 main portion of connection arm
12a end portion of connection arm
12b end portion of connection arm
15 hinges
20 radiation emitting unit(s)/panel
20a . . . 20e radiation emitting units
21 radiation output region
22 connection means
24 hinges
25 radiation source(s)
26 ventilation slot
28a handle
28b handle
29a . . . 29d handles
30 positioning arm
32a first end
32b second end
34 gas spring
35 attachment element
36 first connection means
37a, 37b connecting pipe
38 second connection means
40 radiation source carrier
42 motor
44 carrier edges
46 distance sensor
50 irradiation object cooling system
52 cooling gas driver/fan
54 joint connection
62a gas driver
62b gas driver
63 heat sinks
64 ventilation slot
66 ventilation grate
70a cooling gas outlet
70b cooling gas outlet
100 illumination device
110 common support
110a top end portion
112 foot element
114 wheels
200 irradiation object
300 object location
a1 . . . a6 direction arrows
A1, A2 axis
G1 . . . G3 radiation sources groups
S1 . . . S4 method steps

1. Illumination device for photodynamic therapy, the illumination device comprising at least two electromagnetic radiation emitting units,
the at least two electromagnetic radiation emitting units comprising at least one electromagnetic radiation source,
the electromagnetic radiation source being configured to generate radiation for the irradiation of a region of an irradiation object in an illumination session,
wherein the irradiation object is to be arranged at an object location,
wherein the object location is arranged at a distance relative to a radiation output region of the radiation emitting units through which the radiation generated by the at least one electromagnetic radiation source exits the radiation emitting units during operation of the illumination device.

2. Illumination device according to claim 1, wherein the illumination device comprises an electronic control unit which is configured to control the operation of the illumination device, wherein the respective radiation emitting units are operatively coupled to the electronic control unit, and/or
wherein the illumination device is configured to irradiate an irradiation region of the irradiation object with a predetermined radiation dose of radiation, in particular of radiation with an irradiation wavelength, in one illumination session, and/or
wherein the illumination device is configured to consider temperature dependent variations in the wavelength of the radiation emitted by the radiation sources and/or temperature dependent variations in the optical output power for adjusting parameters of the illumination session in order to irradiate the predetermined radiation dose onto the irradiation region,
and/or
wherein the illumination device comprises at least one temperature sensor, which is configured to measure a temperature characteristic for the temperature of the radiation source(s), wherein the at least one temperature sensor is operatively connected to the electronic control unit to provide temperature data to the electronic control unit, wherein the electronic control unit is configured to adjust the operation of the illumination device based on the temperature data to ensure that the predetermined radiation dose is delivered to the irradiation region.

3.-5. (canceled)

6. Illumination device according to claim 1, wherein the illumination device comprises a radiation sensor which is arranged to receive radiation emitted from the illumination device in order to generate radiation data which is characteristic for a wavelength shift of the peak wavelength of the radiation source, wherein the electronic control unit is configured to adjust the operation of the illumination device based on the radiation data to ensure that the predetermined radiation dose is delivered to the irradiation region, wherein optionally
the radiation received by the radiation sensor is radiation reflected by the irradiation object.

7. (canceled)

8. Illumination device according to claim 1, wherein the operation of the illumination device is adjusted by using one of, an arbitrary combination of or all of the following measures:
varying the distance between the respective radiation emitting unit and the irradiation object,
adjusting the radiation power emitted by the respective radiation emitting unit, and/or
adjusting a duration of the illumination session.

9. Illumination device according to claim 1, wherein the radiation emitting units are connected to a common support via a mechanical connection system, and optionally:
a) wherein the mechanical connection system comprises a connection arm, which is rigidly and/or axially connected to two different radiation emitting units which are movable relative to one another and connected with one another and optionally:
a1) wherein the connection arm has a U-like shape, V-like shape or C-like shape and/or
a2) wherein the connection arm has an adaptable length in order to adapt to different positions which the radiation emitting units assume relative to one another when moved relative to each other.

10.-12. (canceled)

13. Illumination device according to claim 9, option a), wherein at least one radiation emitting unit is arranged between the two radiation emitting units to which the connection arm is connected and/or wherein the radiation emitting units are tiltable relative to the connection arm along an axis which is oblique relative to the axis along which interconnected radiation emitting units are movable relative to one another.

14. (canceled)

15. Illumination device according to claim 1, wherein one or more radiation emitting units are provided with handles and optionally wherein the handles are disposed as different locations on two radiation emitting units.

16. (canceled)

17. Illumination device according to claim 9, option a), wherein
a) two sections of the connection arm which are movable relative to one another are provided with a respective handle, and/or
b) wherein the connection arm is moveable manually and/or by a motor of the illuminating device, and/or
c) wherein the connection arm is movably connected to a positioning arm of the illumination device, the positioning arm being movable connected to the common support and optionally:
c1) wherein the positioning arm is adapted to be connected to or to be connectable to the connection arm on a first end and to a common support of the illumination device on a second end, and/or
c2) wherein the positioning arm is pivotable with respect to a longitudinal axis of the common support of the illumination device, and/or
c3) wherein the first end of the positioning arm comprises first connection means to connect and hold the common support and optionally wherein the first connection means comprises a joint connection, such as to permit movement of the common support with respect to the positioning arm.

18.-23. (canceled)

24. Illumination device according to claim 17, option c), wherein
a) the second end of the positioning arm comprises second connection means, to connect the positioning arm to the common support of the illumination device and optionally wherein: the second connection means comprises a joint, such as to permit independent movement of the positioning arm with respect to the common support of the illumination device, and/or
b) wherein the positioning arm is a spring arm;
and/or
c) further comprising a gas spring, which is operatively connected to the positioning arm to support the positioning arm.

25.-27. (canceled)

28. Illumination device according to claim 1, wherein the illumination device comprises an irradiation object cooling system, and optionally
a) wherein the irradiation object cooling system comprises at least one cooling gas outlet which is configured to face towards the object location wherein the illumination device is configured such that cooling gas can leave the illumination device through the at least one cooling gas outlet and optionally
a1) wherein the illumination device, preferably the respective radiation emitting unit, comprises a cooling gas driver system comprising at least one cooling gas driver, wherein the cooling gas driver system is configured to drive a cooling gas flow through the cooling gas outlet, and optionally
a2) wherein optionally the cooling gas driver is a fan.

29.-31. (canceled)

32. Illumination device according to claim 28, option a), wherein at least one of the radiation emitting units comprises at least one cooling gas outlet, which faces towards the object location
and/or
wherein at least one radiation emitting unit comprises at least two cooling gas outlets, which, preferably, are disposed in opposite end regions of the radiation emitting unit, e.g. separated along the main longitudinal axis.

33. (canceled)

34. Illumination device according to claim 28, option a1), wherein
a) the radiation source carrier comprises one or more cooling gas passages for the cooling gas flow from one side of the radiation source carrier to the opposite side of the radiation source carrier, and optionally:
a1) wherein each cooling gas passage forms one cooling gas outlet or each cooling gas passage is fluidly connected to at least one cooling gas outlet;
and/or
a2) wherein one or more cooling gas passages for the cooling gas flow from one side of the radiation source carrier to the opposite side of the radiation source carrier are arranged adjacent to an edge laterally delimiting the radiation source carrier;
and/or
b) wherein the illumination device is configured such that the cooling gas flows from a side of the radiation source carrier remote from the cooling gas outlet towards the cooling gas outlet.

35.-37. (canceled)

38. Illumination device according to claim 1, wherein the radiation source carrier forms a cooling gas barrier and is closed,
and/or
wherein the illumination device comprises an active radiation source cooling system and optionally:
a) wherein the radiation source cooling system comprises a gas driver configured to move radiation source cooling gas relative to the radiation sources, and/or
b) wherein the active cooling system comprises one or more gas drivers.

39.-41. (canceled)

42. Illumination device according to claim 28, option a), wherein the temperature of the cooling gas at the cooling gas outlet is greater than the ambient temperature and less than or equal to the temperature at a radiation source or a radiation cooling body which is thermally connected to one or more radiation sources of the radiation emitting unit.

43. Illumination device according to claim 38, option a), wherein the illumination device is configured such that radiation source cooling gas is used as cooling gas for cooling the irradiation object with the irradiation object cooling system.

44. A method for treating a skin disease comprising the following steps:
a) applying a pharmaceutical substance to the surface of the skin in a region which is to be treated;
b) arranging the skin region to be treated in a predetermined object location of the illumination device according to claim 1,
c) irradiating the skin region to be treated with the illumination device;
the method further comprising the optional step of:
adjusting the operation of the illumination device on the basis of temperature dependent variations in wavelength of the emitting radiation and/or on the basis of temperature dependent variations in the optical output power; and/or
further comprising the step of: cooling the skin region to the treated with an irradiation object cooling system.

45.-46. (canceled)

47. A method for operating an illumination device according to claim 1, comprising the steps of:
providing a measurement signal which is indicative for temperature dependent variations in the wavelength of the radiation emitted and/or which is indicative for temperature dependent variations in the optical output power,
generating an operation signal as a function of the measurement signal, said operation signal being configured to cause the illumination device to adjust the wavelength of the radiation emitted by the radiation sources of the illumination device or to call for an adjustment of the wavelength of the radiation emitted by the radiation sources of the illumination device and/or
generating an operation signal as a function of the measurement signal, said operation signal being configured to cause the illumination device to adjust the optical output power of the illumination device or to call for an adjustment of the optical output power of the illumination device.

48. A method for operating an illumination device according to claim 1, comprising the steps of:
providing a measurement signal which is indicative for a temperature at the irradiation region of the irradiation object,
generating an operation signal as a function of the measurement signal, said operation signal being configured to cause the illumination device to adjust the operation of the irradiation object cooling system of the illumination device or to call for an adjustment of the operation of the irradiation object cooling system of the illumination device.

49. Computer program product comprising machine-readable instructions, which, when loaded and executed on a processor, are configured to cause the illumination device to execute the method of claim 47.

50. A computer-readable medium having stored thereon the computer program product according to claim 49.

51. The method for treating a skin disease according to claim 44, wherein the pharmaceutical substance comprises 5-aminolevulinic acid.
